(19) United States
(12) Patent Application Publication
(10) Pub. No.: US 2018/0071129 A1
(43) Pub. Date: Mar. 15, 2018

(54) LUMBAR SUPPORT APPARATUS FOR POWERED EXOSKELETON

(71) Applicant: Steering Solutions IP Holding Corporation, Saginaw, MI (US)

(72) Inventors: Muzaffer Y. Ozsecen, Saginaw, MI (US); Patrik M. Ryne, Midland, MI (US)

(21) Appl. No.: 15/262,684

(22) Filed: Sep. 12, 2016

(51) Int. Cl. A61F 5/02; A61H 3/00

(52) U.S. Cl. CPC A61F 5/028; A61F 2250/0003; A61H 3/00

(57) ABSTRACT

A lumbar support apparatus for a powered exoskeleton includes a support member and a lumbar support member. The support member includes a back portion, a first arm, and a second arm. The back portion has a first surface and a second surface that extends between a back portion first end and a back portion second end. The first arm extends from the back portion first end. The first arm defines a first attachment point for a first leg support. The second arm extends from the back portion second end. The second arm defines a second attachment point for a second leg support. The lumbar support member is disposed on the first surface of the back portion.

### BACKGROUND OF THE INVENTION

Powered exoskeletons are used to aid a user in performing various movements such as walking or standing. The power exoskeleton generates torque at respective joints to either assist or resist the user in performing such movements. The powered exoskeleton interacts with the user's body and the generated torque is mechanically grounded to a user's body part. The mechanical grounding to a user's body part or parts may be inefficient.

### SUMMARY OF THE INVENTION

According to an embodiment of the present disclosure, a lumbar support apparatus for a powered exoskeleton is provided. The lumbar support apparatus includes a support member and a lumbar support member. The support member includes a back portion, a first arm, and a second arm. The back portion has a first surface and a second surface that extends between a back portion first end and a back portion second end. The first arm extends from the back portion first end. The first arm defines a first attachment point for a first leg support. The second arm extends from the back portion second end. The second arm defines a second attachment point for a second leg support. The lumbar support member is disposed on the first surface of the back portion.

According to another embodiment of the present disclosure, a lumbar support apparatus is provided. The lumbar support apparatus includes a lumbar support member. The lumbar support member is disposed on a back portion of a support member that is operatively connected to a powered exoskeleton. The lumbar support member has a base member and a contoured support surface. The base member is disposed on the back portion of the support member. The contoured support surface extends from the base member. The contoured support surface has a first ridge, a second ridge, and a trough disposed between the first ridge and the second ridge.

According to yet another embodiment of the present disclosure, a method of manufacturing a lumbar support member is shown. The method includes positioning a support member having a cavity and a cavity and a membrane disposed over the cavity relative to a lumbosacral region of a user of a power exoskeleton. The method further includes applying a hardenable foam to the cavity.

These and other advantages and features will become more apparent from the following description taken in conjunction with the drawings.

### BRIEF DESCRIPTION OF THE DRAWINGS

The subject matter which is regarded as the invention is particularly pointed out and distinctly claimed in the claims at the conclusion of the specification. The foregoing and other features, and advantages of the invention are apparent from the following detailed description taken in conjunction with the accompanying drawings in which:

FIG. 1 is an environmental view of a powered exoskeleton attached to a user;

FIG. 2 is a perspective view of a lumbar support apparatus attachable to the powered exoskeleton;

FIG. 3 is a top perspective view of the lumbar support apparatus;

FIG. 4 is a top perspective view of a lumbar support member of the lumbar support apparatus;

FIG. 5 is a perspective view of the lumbar support member;

FIG. 6 is a side perspective view of the lumbar support member;

FIG. 7 is a perspective view of a lumbar support member having an inflatable lumbar support member;

FIGS. 8a-10b are illustrative views of a method of manufacturing a lumbar support member;

FIG. 11 is a front perspective view of a powered exoskeleton having an adjustment assembly and a strap assembly; and

FIG. 12 is a rear perspective view of the powered exoskeleton having an adjustment assembly and a strap assembly.

### DETAILED DESCRIPTION

Referring now to the Figures, where the present disclosure will be described with reference to specific embodiments, without limiting same, it is to be understood that the disclosed embodiments are merely illustrative and may be embodied in various and alternative forms. The Figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure.

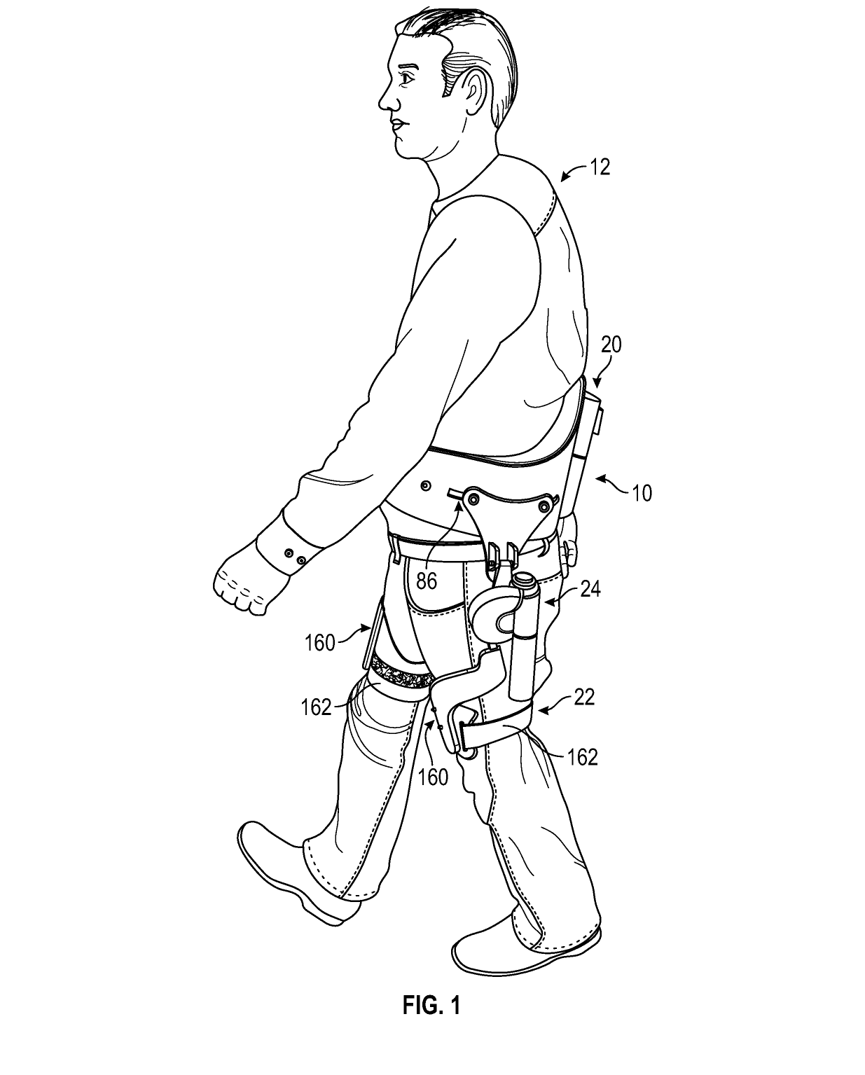

Referring to FIG. 1 an environmental view of a powered exoskeleton 10 that is attachable to a user 12 is shown. The powered exoskeleton 10 is wearable by the user 12 to aid the user 12 in performing various movements, tasks, or to reduce the user's energy consumption during various movements. The powered exoskeleton 10 is mechanically grounded to a portion of the user 12 to aid in the transfer of torque by the powered exoskeleton 10 to the user 12. The powered exoskeleton 10 includes a lumbar support apparatus 20, at least one leg support 22, and an actuator 24.

The lumbar support apparatus 20 is configured as a torso brace that interfaces with the user 12. The lumbar support apparatus 20 is disposed about a user's waist proximate a user's hip region. The lumbar support apparatus 20 is configured to adjust overall human-exoskeleton interface stiffness through the use of various lumbar support types. The various lumbar support types permit the user 12 to adjust for comfort and load or torque transfer efficiency from the powered exoskeleton 10 to the user 12.

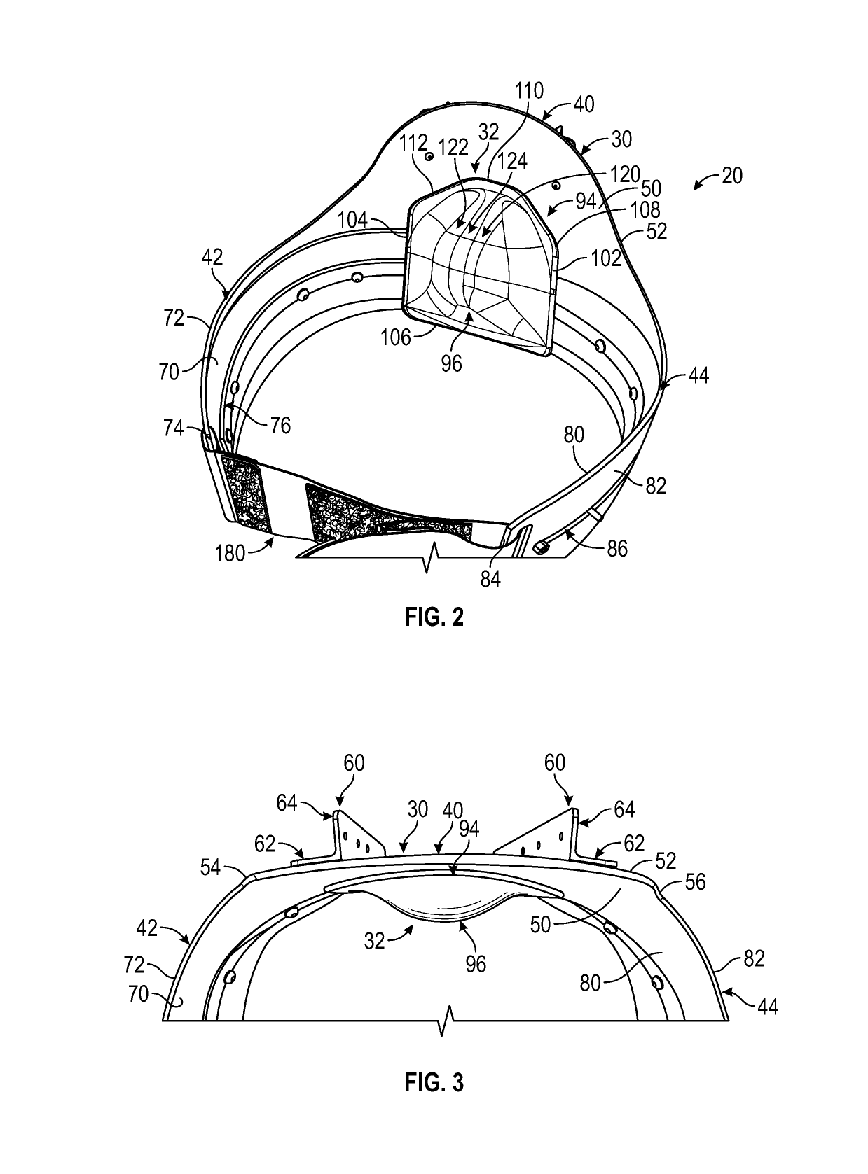

Referring to FIGS. 2 and 3, the lumbar support apparatus 20 includes a support member 30 and a lumbar support member 32. The lumbar support apparatus 20 is geometrically matched with a user 12 to improve load or torque transfer efficiency from the powered exoskeleton 10 to the user 12.

The support member 30 includes a back portion 40, a first arm 42, and a second arm 44. The back portion 40 is disposed proximate a lumbosacral region of a back of the user 12. The back portion 40 has a first surface 50 and a second surface 52 disposed opposite the first surface 50. The first surface 50 and the second surface 52 extend between a back portion first end 54 and a back portion second end 56.

The support member 30 may be operatively connected to the powered exoskeleton 10 by at least one reinforcement member 60 that is disposed on the back portion 40. The at least one reinforcement member 60 is disposed on the second surface 52 of the back portion 40. The at least one reinforcement member 60 is configured to function as a reinforcement or stiffening member of the back portion 40 to aid in load or torque transfer of the powered exoskeleton 10 to the user 12.

The at least one reinforcement member 60 includes a first reinforcement 62 disposed on the second surface 52 of the back portion 40 and a second reinforcement 64 extending from the first reinforcement 62. The first reinforcement 62 is securely attached to the second surface 52 of the back portion 40. The second reinforcement 64 is disposed substantially perpendicular to the first reinforcement 62. The second reinforcement 64 extends away from the second surface 52 of the back portion 40.

The first arm 42 extends from the back portion first end 54. The first arm 42 extends at least partially about the waist of the user 12. The first arm 42 includes a first arm first surface 70 and a first arm second surface 72 disposed opposite the first arm first surface 70. The first arm first surface 70 and the first arm second surface 72 extends between the back portion first end and a first arm end 74. The first arm first surface 70 faces towards the user 12. The first arm second surface 72 faces away from the user 12.

The first arm 42 defines a first attachment point 76 for the at least one leg support 22 and/or the actuator 24. The first attachment point 76 extends from the first arm second surface 72 towards the first arm first surface 70.

The second arm 44 extends from the back portion second end 56. The second arm 44 extends at least partially about the waist of the user 12. The second arm 44 includes a second arm first surface 80 and a second arm second surface 82 disposed opposite the second arm first surface 80. The second arm first surface 80 and the second arm second surface 82 extend between the back portion second end 56 and a second arm end 84. The second arm first surface 80 faces towards the user 12. The second arm second surface 82 faces away from the user 12.

The second arm 44 defines a second attachment point 86 for the at least one leg support 22 and/or the actuator 24. The second attachment point 86 extends from the second arm second surface 82 towards the second arm first surface 80.

The lumbar support member 32 is disposed on the first surface 50 of the back portion 40 of the support member 30. The lumbar support member 32 is configured as a contoured lumbar support that improves the load or torque transfer efficiency from the powered exoskeleton 10 to the user 12.

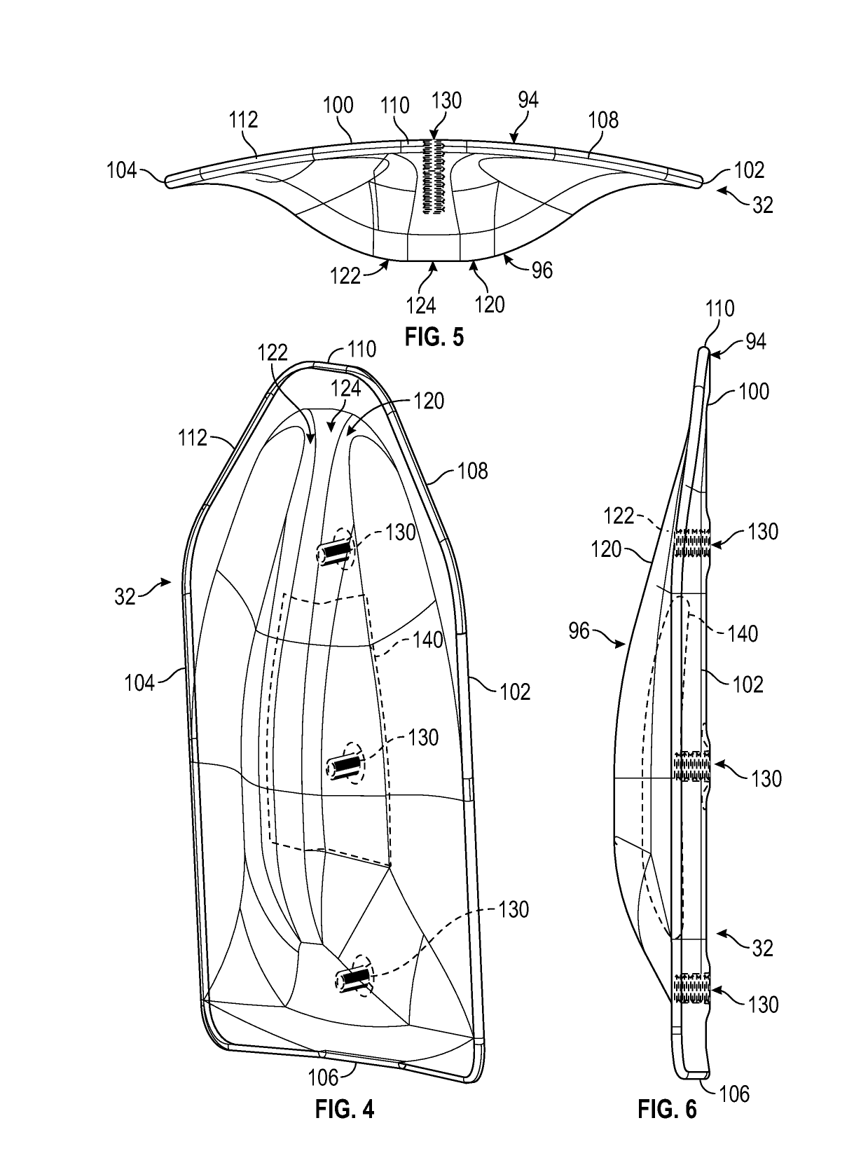

Referring to FIGS. 4-6, the lumbar support member 32 includes a base member 94 and a contoured support surface 96. The base member 94 is disposed on the first surface 50 of the back portion 40. The base member 94 includes a base surface 100 that extends between a first side 102, a second side 104, a third side 106, a fourth side 108, a fifth side 110, and a sixth side 112.

The second side 104 is disposed opposite the first side 102. The third side 106 extends between a distal end of the first side 102 and a distal end of the second side 104. The fourth side 108 extends from a proximal end of the first side 102 towards the fifth side 110. The fifth side 110 is disposed opposite the third side 106. The sixth side 112 extends from the fifth side 110 to a proximal end of the second side 104.

The fourth side 108 and the sixth side 112 become progressively closer to each other in a direction that extends from the third side 106 towards the fifth side 110. The fourth side 108, the fifth side 110, and the sixth side 112 may form a tapered region of the base member 94.

The contoured support surface 96 extends from the base member 94. The contoured support surface 96 is configured to engage the lumbar region of the user 12. More specifically, the contoured support surface 96 is configured to engage a region of a user's back that extends between the users L1 and L5 vertebrae.

The contoured support surface 96 is disposed opposite the base surface 100. The contoured support surface 96 includes a first ridge 120, a second ridge 122, and a trough 124. The first ridge 120, the second ridge 122, and the trough 124 are vertically aligned in a direction that extends from the third side 106 towards the fifth side 110.

The first ridge 120 is configured to engage first paraspinal musculatures disposed proximate a first side of the spine of the user 12. The first ridge 120 has a first surface profile. The first surface profile is configured to match the curvature of the back of the user 12 proximate the lumbar region. The first surface profile has a cambered region that engages the first paraspinal musculatures proximate the L2, L3, and L4 vertebrae of the spine of the user 12 to provide vertical support.

The second ridge 122 is configured to engage the second paraspinal musculatures disposed proximate a second side of the spine of the user 12. The second paraspinal musculatures are disposed opposite the first paraspinal musculatures. The second ridge 122 has a second surface profile. The second surface profile is configured to match the curvature of the back of the user 12 proximate the lumbosacral region. The second surface profile has a cambered region that engages the second paraspinal musculatures proximate the L2, L3, and L4 vertebrae of the spine of the user 12 to provide vertical support.

The trough 124 is disposed between the first ridge 120 and the second ridge 122. The trough 124 is proximately aligned with the spine of the user 12 such that the spine of the user 12 is disposed between the first ridge 120 and the second ridge 122.

The base surface 100 defines at least one fastener opening 130. The at least one fastener opening 130 extends from the base surface 100, through the base member 94, towards the contoured support surface 96. The at least one fastener opening 130 is proximately aligned with the trough 124 and is disposed between the first ridge 120 and the second ridge 122. The at least one fastener opening 130 is configured to receive a fastener to couple the lumbar support member 32 to the support member 30.

In at least one embodiment, the lumbar support member 32 defines a cavity 140 that is disposed between the base member 94 and the contoured support surface 96. The cavity 140 is spaced apart from the at least one fastener opening 130. The at least one fastener opening 130 may extend into the cavity 140.

The cavity 140 may be configured to receive power electronics or control devices. The power electronics or control devices may be communication with the actuator 24 and configured to operate the actuator 24 and the at least one leg support 22.

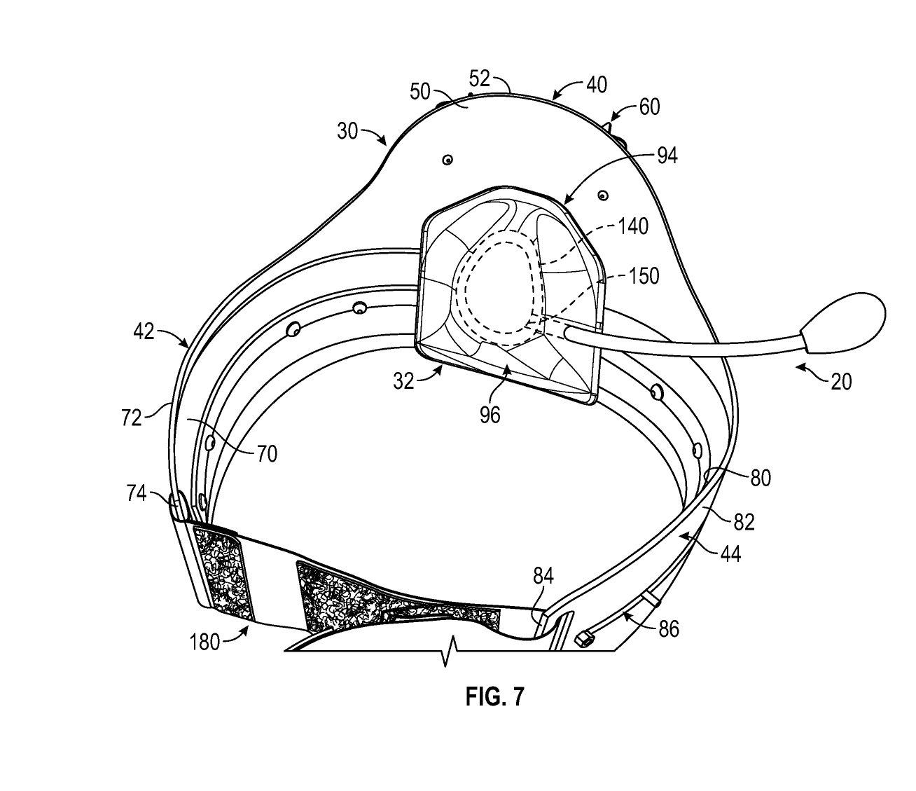

Referring to FIG. 7, the cavity 140 of the lumbar support member 32 is configured to receive an inflatable member 150, such as an air bladder. The inflatable member 150 is operatively connected to a pneumatic system 152. The pneumatic system 152 may be configured as ball or bulb type pump fluidly connected to the inflatable member 150 through a connecting hose. The pneumatic system 152 is configured to selectively provide a working fluid, such as air, to the inflatable member 150 to vary a stiffness of an interface between the user 12 and the contoured support surface 96 of the lumbar support member 32. The inflatable member 150 may be actively controlled by the user 12 to provide an adjustable level of comfort to the user 12. The lumbar support member 32 having an inflatable member 150 is configured to provide rigidity that is less than a rigidity of the lumbar support member 32 without the inflatable member 150.

Referring to FIG. 1, the at least one leg support 22 is attached to at least one of the first attachment point 76 or the second attachment point 86 of the support member 30. The at least one leg support 22 is configured to interface with a lower extremity of the user 12 such as a lower limb of the user 12. The at least one leg support 22 includes a linkage system 160 that is secured to the lower limb of the user 12 by interface members 162, such as straps, bars, brackets, elastomers, or the like. The linkage system 160 facilitates flexion and extension of the lower limb of the user 12.

The actuator 24 is configured to interface with the at least one leg support 22. The actuator 24 may extend between the lumbar support apparatus 20 and the at least one leg support 22. In at least one embodiment, the actuator 24 is directly connected to at least one of the first attachment point 76 or the second attachment point 86. The actuator 24 is configured to receive power from a power unit to create torque or load to aid the user 12 in movement. The actuator 24 may be an electric motor, a stepping motor, a hydraulic actuator, a pneumatic actuator, or the like.

The lumbar support apparatus 20 is configured to improve the load or torque transfer efficiency from the actuator 24 to the at least one leg support 22 by more securely positioning the lumbar support apparatus 20 relative to the lumbosacral region of the user 12. The geometric contouring or shape matching of the lumbar support member 32 with the overall shape of the spine lumbar section proximate the lumbosacral region of the back of the user 12 minimizes or inhibits relative motion of at least one of the support member 30 and the lumbar support member 32 relative to the user 12. The inhibition or minimization of relative motion of at least one of the support member 30 and the lumbar support member 32 relative to the user 12 improves the load or torque transfer efficiency of the powered exoskeleton 10.

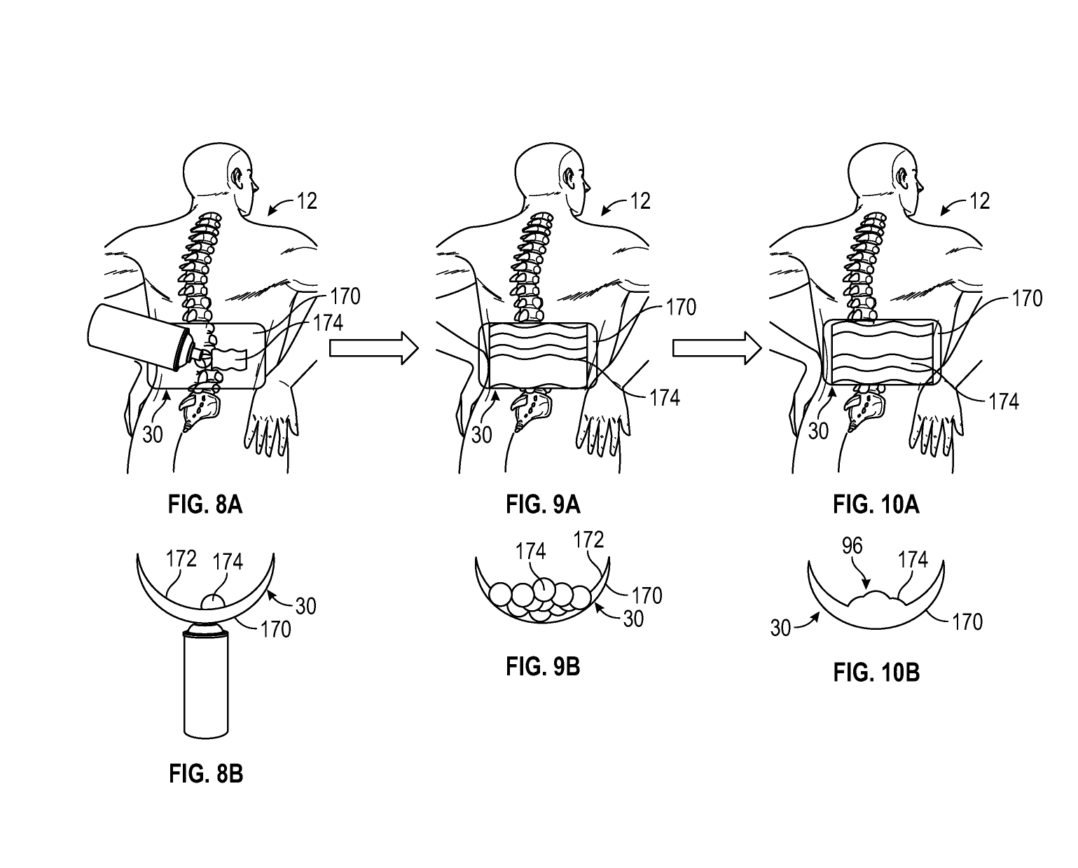

Referring to FIGS. 8a-10b, an illustrative method of manufacturing a lumbar support member is shown. Referring to FIGS. 8a and 8b, the support member 30 is provided with a cavity 170 having a membrane 172 disposed over the cavity 170. The cavity 170 extends into or extends from the back portion 40 of the support member 30. The support member 30 having a cavity 170 is positioned relative to the lumbosacral region of the user 12 of the powered exoskeleton 10. The membrane 172 may engage the back of the user 12 of the powered exoskeleton 10.

Hardenable foam 174 may be applied to or sprayed into the cavity 170. The hardenable foam 174 may be sprayed into a region that is disposed between the cavity 170 and the membrane 172.

Referring to FIGS. 9a and 9b, the hardenable foam 174 expands within the region that is disposed between the cavity 170 and a membrane 172 such that the hardenable foam 174 at least partially fills the cavity 170. The hardenable foam 174 fills up a gap that is disposed between the user 12 and the support member 30. The hardenable foam 174 takes the form of the shape of the back of the user 12 proximate the lumbosacral region. Upon the hardenable foam 174 taking an initial shape, the support member 30 having the hardenable foam 174 may be separated from the user 12 and be left to cure.

Referring to FIGS. 10a and 10b, the hardenable foam 174 is cured for a predetermined period of time and forms the contoured support surface 96. Upon curing the contoured support surface 96 for the predetermined period of time, a custom lumbar support apparatus is now ready for use with the powered exoskeleton 10.

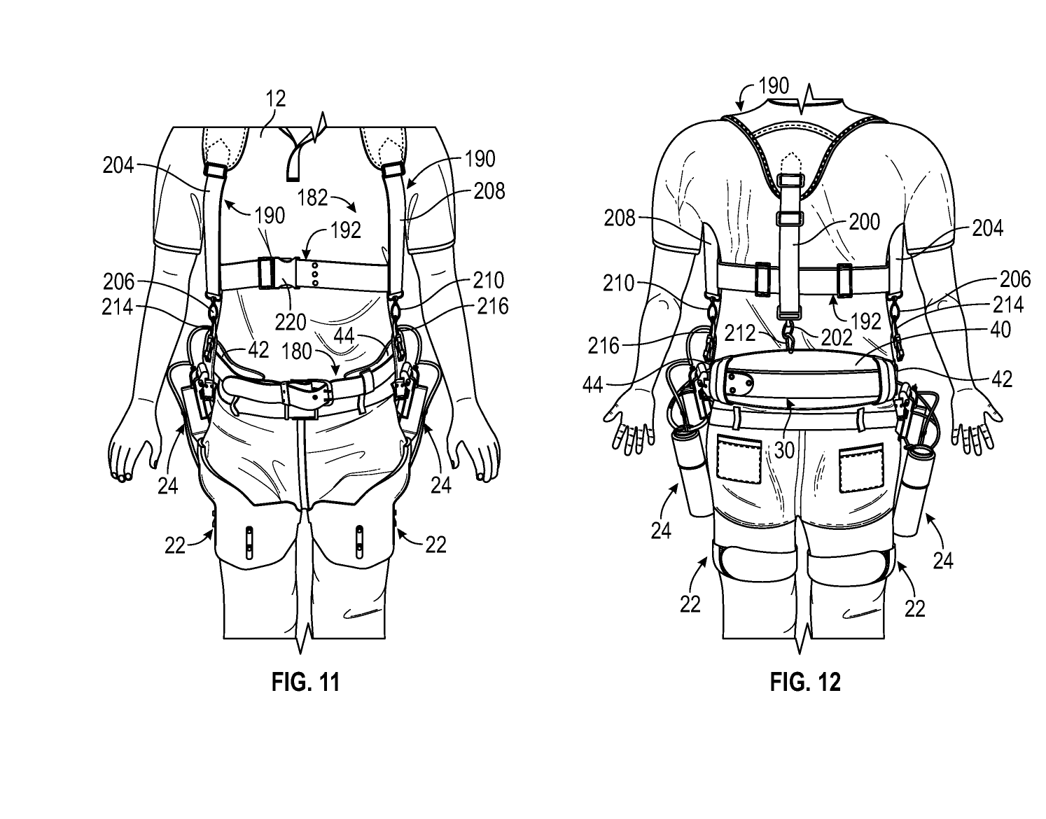

The powered exoskeleton 10 may be provided with an adjustment assembly 180 and/or a strap assembly 182. The adjustment assembly 180 extends between the first arm end 74 of the first arm 42 and the second arm end 84 of the second arm 44. The adjustment assembly 180 is configured to adjustably secure the support member 30 and the lumbar support member 32 to the user 12. The adjustment assembly 180 includes at least one strap provided with a strap keeper that permits the user 12 to adjust the effective length of the at least one strap of the adjustment assembly 180 to provide a desired tension about the waist of the user 12, as shown in FIG. 11. The strap may be an elastic strap. The adjustment assembly 180 may be configured as a belt and a buckle or other fastener to adjust tension about the waist of the user 12. The belt may be a leather or fabric belt.

Referring to FIGS. 11 and 12, the strap assembly 182 includes a shoulder strap assembly 190 and a thorax strap assembly 192. The shoulder strap assembly 190 includes an anchor strap 200 having a first connector 202, a first shoulder strap 204 having a second connector 206, and a second shoulder strap 208 having a third connector 210. The first connector 202 of the anchor strap 200 is connected to the back portion 40 of the support member 30. The first connector 202 of the anchor strap 200 is connected to a first mating connector 212 that is operatively connected to or extends from the back portion 40 of the support member 30. The first connector 202 may be configured as clamp, hook, or the like and the first mating connector 212 may be configured as a ring, hoop, or the like that is configured to receive the first connector 202.

The anchor strap 200 having the first connector 202 extends from the back portion 40 of the support member 30 that is disposed proximate the lumbosacral region towards the thoracic (mid-thoracic) region of the user's back. In at least one embodiment, a shoulder pad is provided that extends from an end of the anchor strap 200 over the user's shoulders. The first shoulder strap 204 having the second connector 206 and the second shoulder strap 208 having the third connector 210 each extend from the anchor strap 200 having the first connector 202 and extend over the user's shoulders or extend from the shoulder pad.

The first shoulder strap 204 having the second connector 206 extends towards the first arm 42 of the support member 30 of the lumbar support apparatus 20. The second connector 206 of the first shoulder strap 204 is connected to a second mating connector 214 that is operatively connected to or extends from the first arm 42 of the support member 30 proximate the anterior-superior iliac spinal region of the user 12. The second connector 206 may be configured as clamp, hook, or the like and the second mating connector 214 may be configured as a ring, hoop, or the like that is configured to receive the first connector 202.

The second shoulder strap 208 having the third connector 210 extends towards the second arm 44 of the support member 30 of the lumbar support apparatus 20. The third connector 210 of the second shoulder strap 208 is connected to a third mating connector 216 that is operatively connected to or extends from the second arm 44 of the support member 30 proximate the anterior-superior iliac spinal region of the user 12. The third connector 210 may be configured as clamp, hook, or the like and the third mating connector 216 may be configured as a ring, hoop, or the like that is configured to receive the first connector 202.

The thorax strap 192 is operatively connected to the anchor strap 200 and extends about the torso of the user 12 proximate the thoracic region of the user 12. The thorax strap 192 is configured to adjustably secure the support member 30 of the lumbar support apparatus 20 to the user 12. The thorax strap 192 may be an elastic strap, a leather strap, or fabric strap. Ends of the thorax strap 192 may be secured to each other by a securing mechanism 220 such as clips, fasteners, buckles, or the like.

Throughout this specification, the term “attach,” “attachment,” “connected”, “coupled,” “coupling,” “mount,” or “mounting” shall be interpreted to mean that one structural component or element is in some manner connected to or contacts another element—either directly or indirectly through at least one intervening structural element—or is integrally formed with the other structural element.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the scope of the invention. Additionally, while various embodiments of the invention have been described, it is to be understood that aspects of the invention may include only some of the described embodiments. Accordingly, the invention is not to be seen as limited by the foregoing description.

Having thus described the invention, it is claimed:

1. A lumbar support apparatus for a powered exoskeleton, comprising:
a support member including
a back portion having a first surface and a second surface extending between a back portion first end and a back portion second end,
a first arm extending from the back portion first end, first arm defining a first attachment point for a first leg support, and
a second arm extending from the back portion second end, the second arm defining a second attachment point for a second leg support; and
a lumbar support member disposed on the first surface of the back portion.

2. The lumbar support apparatus of claim 1, wherein the lumbar support member includes a base member having a first side, a second side disposed opposite the first side, a third side extending between the first side and the second side, a fourth side extending from the first side towards a fifth side, and a sixth side extending towards the fifth side.

3. The lumbar support apparatus of claim 2, wherein the fourth side and the sixth side become progressively closer to each other in a direction that extends towards the fifth side.

4. The lumbar support apparatus of claim 2, wherein the lumbar support member further includes a base surface that extends between the first side, the second side, the third side, the fourth side, the fifth side, and the sixth side that is configured to engage the first surface.

5. The lumbar support apparatus of claim 4, wherein the lumbar support member further includes a contoured support surface disposed opposite the base surface.

6. The lumbar support apparatus of claim 5, wherein the lumbar support member defines at least one fastener opening that extends from the base surface towards contoured support surface.

7. The lumbar support apparatus of claim 5, wherein the contoured support surface includes a first ridge having a first surface profile and a second ridge having a second surface profile.

8. The lumbar support apparatus of claim 7, wherein the contoured support surface includes a trough disposed between the first ridge and the second ridge.

9. The lumbar support apparatus of claim 8, wherein the first ridge is configured to engage first paraspinal musculatures disposed proximate a first side of a user's spine and the second ridge is configured to engage second paraspinal musculatures disposed proximate a second side of a user's spine.

10. The lumbar support apparatus of claim 1, further comprising an adjustment assembly extending between the first arm and the second arm, the adjustment assembly configured to adjustably secure the support member to a user.

11. A lumbar support apparatus, comprising:
a lumbar support member disposed on a back portion of a support member operatively connected to a powered exoskeleton, the lumbar support member including:
a base member disposed on the back portion of the support member; and
a contoured support surface extending from the base member, the contoured support surface includes a first ridge, a second ridge, and a trough disposed between the first ridge and the second ridge.

12. The lumbar support apparatus of claim 11, wherein the first ridge and the second ridge are configured to engage a region of a user's back that extends between a user's L1 and L5 vertebrae.

13. The lumbar support apparatus of claim 11, wherein the lumbar support member defines a cavity disposed between the base member and the contoured support surface.

14. The lumbar support apparatus of claim 13, wherein the cavity is configured to receive an inflatable member.

15. The lumbar support apparatus of claim 14, wherein the inflatable member is operatively connected to pneumatic system configured to selectively provide a working fluid to the inflatable member to vary interface stiffness between a user and the powered exoskeleton.

16. A method of manufacturing a lumbar support member, comprising:
positioning a support member having a cavity and a membrane disposed over the cavity relative to a lumbosacral region of a user of a powered exoskeleton; and
applying a hardenable foam to the cavity.

17. The method of claim 16, further comprising:
expanding the hardenable foam within the cavity to form a contoured support surface having a first ridge that engages first paraspinal musculatures disposed proximate a first side of a user's spine, a second ridge that engages second paraspinal musculatures disposed proximate a second side of a user's spine, and a trough disposed between the first ridge and the second ridge.

18. The method of claim 17, further comprising:
curing the contoured support surface for a predetermined period of time.
